(19) United States
(12) Patent Application Publication
(10) Pub. No.: US 2012/0265032 A1
(43) Pub. Date: Oct. 18, 2012

(54) OVULATING SENSING AND ANALYZING SYSTEM

(75) Inventors: Yoav Ben-David, Tel Aviv (IL); Alfonso Rodriguez, Zurich (CH); Boaz Mendel, Jerusalem (IL); Boaz Beeri, Jerusalem (IL)

(73) Assignee: Arie FRIDENSON, Haifa (IL)

(21) Appl. No.: 13/514,469

(22) PCT Filed: Dec. 9, 2010
(86) PCT No.: PCT/IL10/01038

§ 371 Date: Jun. 7, 2012

Related U.S. Application Data
(60) Provisional application No. 61/267,971, filed on Dec. 9, 2009.

(51) Int. Cl. A61B 5/01; A61B 5/00

(52) U.S. Cl. 600/301; 600/549

(57) ABSTRACT

The present invention relates to a system for measuring an oral temperature of a female subject during an ovulation cycle of the female subject for assisting the female subject in determining the next ovulation cycle for fertility and/or birth-control and includes a method and apparatus for predicting a commencement of a new ovulation cycle in a female subject including measuring and communicating the data to a remote computing platform by daily measuring the oral temperatures and calculating an average oral temperature for an ovulation cycle. The daily oral temperature measurements and/or average oral temperatures are also available on external databases and may be used for comparing and checking the current temperature measurements. Different sensors are available for other purposes.

### FIELD OF THE INVENTION

The present invention relates to a system for measuring an oral temperature of a female subject during an ovulation cycle of the female subject for assisting the female subject in determining the next ovulation cycle for fertility and/or birth-control.

### BACKGROUND OF THE INVENTION

The following prior art are believed to be relevant to the current status of the art:

U.S. Pat. No. 4,148,304 describes a system for determining the time of ovulation in females including two probes one of which is inserted into the female's ear (FIG. 2) and the second probe is inserted on the forehead of the female (FIG. 4). The prior art system measures the voltage potential between the two probes as well as the body temperature. The prior art system is drawn to determining the time of ovulation.

U.S. Pat. No. 5,137,028 describes a clinical thermometer for women. The thermometer has processing functions to determine ovulation days and also a function for memorizing the data for several menstruation periods and to display the data when desired to guide exact diagnosis.

EP 0 476 730 A1 describes a system for ovulation detection and prediction by implanting a temperature sensor into the cervix of the female and measuring the basal body temperature over a predetermined interval of time. The system is removed from the cervix for processing at the end of the time interval.

CN 2586146Y describes a digital thermometer for measuring the basal body temperature. The thermometer is located on a body and connected to a controller unit which interfaces with a microcomputer.

The previous prior art is inconvenient and invasive for a woman to use, either for fertility control or for contraception. There is thus a long felt and unmet need for a convenient and accurate method for fertility control or for contraception.

### SUMMARY OF THE INVENTION

The previous prior art does not consider fertility control and birth control based on oral temperature measurements. Thus, there is a need in the art for a mobile device which provides couples with an indication of the chances of a woman partner becoming pregnant or for the woman partner not becoming pregnant.

It is an acknowledged purpose of the present invention to provide devices and methods for increasing the chances of a woman to become pregnant.

It is an acknowledged purpose of the present invention to provide devices and methods for avoiding pregnancy by means of non invasive devices and methods of contraception.

The present invention seeks to provide a female subject with a prediction of a commencement of her ovulation cycle by measuring the oral temperature of the female subject with a mobile oral temperature sensing system. The results of the temperature measurements are communicated to a remote computing platform. The remote computing system includes a processor which compares the current oral temperature measurements with previous stored oral temperature measurements thus providing the female subject a mobile pregnancy indicator and/or a mobile contraceptive indicator.

In the present invention, the female subject measures her oral temperature during the ovulation period and communicates the measured oral temperature of the female subject to a remote computer device. At the remote computing platform, the measured oral temperature is recorded, stored, analyzed and compared to previous oral temperature measurements to enable the female subject to predetermine and predict the date of a next ovulation cycle.

The present invention provides a female subject with a mobile predictor device for assisting the female subject to determine the next ovulation cycle for either fertility and/or for birth-control, thus, indicating to a couple the chances of the female partner becoming pregnant or not becoming pregnant.

In the present invention, the female subject measures her oral temperature daily over a predetermined time interval, such as a 12-day time interval with an oral sensor device. The oral sensor device communicates the measured oral temperatures to a remote computing platform for recording, storing and analyzing the measured oral temperature on a display unit. The computing platform also compares the current oral temperature measurement with previous measurements in order to enable the female subject to predetermine and predict the commencement of the next ovulation cycle.

The communication of the oral temperature measurements are communicated from the temperature sensing device to the remote computer device by communications means, such as RF transmissions communications systems, for example, via a cellular communications network, a Bluetooth communications system and an IR communications system.

It is an object of this invention to meet the long-felt and unmet needs of predicting the date of commencement of an ovulation cycle. In particular, it is an object of the present invention to disclose a method for predicting a commencement of a new ovulation cycle in a female subject including recording the commencement and termination of a menstruation cycle, measuring on a daily basis, an oral temperature of said female subject during a current ovulation cycle following said termination of said menstruation cycle, calculating a current running average of the daily temperature measurements, comparing each daily measurement with at least one previous average oral temperature calculated from at least one previous ovulation cycle, storing said current daily measured oral temperatures, storing said current daily running average temperatures of current measured oral temperatures, calculating a current average oral temperature from said current daily oral temperature measurements, storing said current average oral temperature, comparing and analyzing said current daily temperature with said at least one previous average temperature, predicting said commencement of said new ovulation cycle in said female subject based on said analysis.

It is a further object of this invention to disclose such a method including comprising checking if each daily temperature measurement of said oral temperature measurements is within a predetermined temperature range of said previous average temperature and within said running average temperature.

It is a further object of this invention to disclose such a method including cancelling said daily temperature measurement if said daily oral temperature measurement of said oral temperature measurements exceeds said predetermined said previous average temperature and said running average temperature, alerting said female subject that the current daily temperature exceeds said predetermined temperature range, repeating said daily oral temperature measurement to obtain a new daily oral temperature measurement, calculating a further average current oral temperature, analyzing said further set of current oral temperature measurements with previously stored oral temperature measurements, and predicting a commencement of a new ovulation cycle of said female subject based on said analysis.

It is a further object of this invention to disclose such a method wherein the oral temperature measurements are measured over a 12-day time interval.

It is a further object of this invention to disclose such a method including predicting the sex of a conceived child.

It is a further object of this invention to disclose that such a method is noninvasive.

It is a further object of this invention to disclose such a method wherein the predetermined temperature range is 0.15-0.45° C.

It is a further object of this invention to disclose an ovulation sensing and analyzing system for measuring oral temperatures of a female subject comprising an oral temperature sensing system, said oral temperature sensing system including at least one sensing device, and a remote computing platform. The at least one sensing device includes an oral temperature measuring sensor to measure said oral temperatures and said at least one sensing device communicates said measured temperatures to said remote computing platform and said remote computing platform analyzes said oral temperature measurements to predict and display on an output display device a date of a next ovulation cycle of said female subject.

It is a further object of this invention to disclose such a system includes at least one sensing device further comprising a control unit, said control unit includes an A/D device for converting measured oral temperatures analog signals to digital signals, and a first transceiver for modulation and communicating said digital signals to said remote computing device.

It is a further object of this invention to disclose such a system includes the oral temperature sensor is selected from any one of a group consisting of a thermometer, an oral thermocouple device, a saliva-sensitive temperature measuring device, a digital thermometer, a thermometer sensitive to a predetermined range of temperatures or any combination thereof.

It is a further object of this invention to disclose such a system includes a transceiver for demodulating said communicated signals, a processor for processing said demodulated signals, and an output device for displaying said processed signals.

It is a further object of this invention to disclose such a system includes at least one switch device for activating said at least one sensing device to commence oral temperatures measurements and to communicate said oral temperature measurements to said remote computing platform.

It is a further object of this invention to disclose such a system includes the oral temperature sensing system is selected from any one of a group consisting of a smart cellular phone, a laptop, a PDA device or any combination thereof.

It is a further object of this invention to disclose such a system includes a remote computing platform analyzes and displays said analyzed data in graphical form.

It is a further object of this invention to disclose such a system includes at least one sensing device includes a USB connection interface for coupling said at least one sensing device to an external storage and analysis device.

It is a further object of this invention to disclose such a system includes wherein an oral temperature sensing system detects and measures temperature changes in a range of 0.15-0.45° C.

It is a further object of this invention to disclose such a system includes an audio sensor for sensing audio data for measuring environmental noise.

It is a further object of this invention to disclose such a system includes a virus detector for detecting virus data.

It is a further object of this invention to disclose such a system includes a a sensor for detecting hormonal changes in a body.

It is a further object of this invention to disclose such a system includes a body fat-muscle relation detecting sensor for sensing the body fat-muscle relation data.

It is a further object of this invention to disclose such a system includes a sensor for sensing and measuring ambient temperature.

It is a further object of this invention to disclose such a system includes a wristwatch including a sensor for detecting and storing body temperatures.

It is a further object of this invention to disclose such a system also includes a key ring for carrying said temperature sensing device.

It is a further object of this invention to disclose a method for measuring an oral temperature of a female subject during an ovulation cycle of a female subject, including providing an oral temperature sensor, measuring said oral temperature with said oral temperature sensor, comparing and analyzing said measured oral temperatures and previously stored oral temperatures of said female subject, and predicting a date of a next ovulation cycle of said female subject.

It is a further object of this invention to disclose such a method including displaying said next ovulation cycle on a display device.

It is a further object of this invention to disclose such a method for including an A/D device for converting measured oral temperatures to digital signals, and a first transceiver for modulation and communicating said digital signals to said computing device.

It is a further object of this invention to disclose a method including providing an oral temperature sensor selected from any one of a group consisting of a thermometer, an oral thermocouple device, a saliva-sensitive temperature measuring device, a digital thermometer, a thermometer sensitive to a predetermined range of temperatures or any combination thereof.

It is a further object of this invention to disclose such a method wherein the remote computing platform receiving and demodulating said communicated signals, processing said demodulated signals, and displaying said processed signals on said display device.

It is a further object of this invention to disclose such a method including providing at least one switch device for activating said oral temperature sensing device to commence oral temperatures and to communicate said temperature measurements to said remote computing platform.

It is a further object of this invention to disclose such a method including selecting said oral temperature sensing system from any one of a group consisting of a smart cellular phone, a laptop, a PDA device or any combination thereof

It is a further object of this invention to disclose such a method including providing said remote computing platform for analyzing and displaying said analyzed data in graphical form.

It is a further object of this invention to disclose such a method including a USB connection interface located on said sensing device for coupling said sensing device to an external storage and analysis device.

It is a further object of this invention to disclose such a method including providing said oral temperature sensing device for detecting and measuring temperature changes in a range of 0.15-0.45° C.

It is a further object of this invention to disclose such a method including providing an audio sensor for sensing audio data for measuring environmental noise.

It is a further object of this invention to disclose such a method including providing a virus detector for detecting virus data.

It is a further object of this invention to disclose such a method including providing a sensor for detecting hormonal changes in a body.

It is a further object of this invention to disclose such a method including providing a sensor for sensing body fat-muscle relation sensor and the sensed data is the body fat-muscle relation.

It is a further object of this invention to disclose such a method including providing a body fat-muscle relation detecting sensor for sensing the body fat-muscle relation data.

It is a further object of this invention to disclose such a method including providing a wristwatch including a sensor for detecting and storing body temperature.

It is a further object of this invention to disclose such a method for including providing a key ring for carrying said temperature sensing device.

It is a further object of this invention to disclose such a method including comparing and analyzing includes at least one iterative step for predicting said commencement of said new ovulation cycle in said female subject.

It is a further object of this invention to disclose such a method including comparing and analyzing at least one learning step for predicting said commencement of said new ovulation cycle in said female subject.

It is a further object of this invention to disclose such a method including comparing and analyzing includes at least one feedback step for predicting said commencement of said new ovulation cycle in said female subject.

It is a further object of this invention to disclose such a method including at least one sensing device is selected from any one of a group consisting of a temperature sensor, a microbiological sensor, a gas analysis sensor, a pH sensor, an electroconductivity sensor, a physiological sensor, a biometric sensor, a miniature stills camera or video imaging device with or without illumination means such as but not limited to an LED, a fiber optic device, or any combination thereof.

It is a further object of this invention to disclose that such a method includes at least one of said sensors is noninvasive.

It is a further object of this invention to disclose that such a method is noninvasive.

It is a further object of this invention to disclose such a method wherein the predetermined temperature range is 0.15-0.45° C.

In accordance with a preferred embodiment of the present invention, a method for predicting a commencement of a new ovulation cycle in a female subject including recording the commencement and termination of a menstruation cycle, measuring on a daily basis, an oral temperature of said female subject during a current ovulation cycle following said termination of said menstruation cycle, calculating a current running average of the daily temperature measurements, comparing each daily measurement with at least one previous average oral temperature calculated from at least one previous ovulation cycle, storing said current daily measured oral temperatures, storing said current daily running average temperatures of current measured oral temperatures, calculating a current average oral temperature from said current daily oral temperature measurements, storing said current average oral temperature, comparing and analyzing said current daily temperature with said at least one—previous average temperature and predicting said commencement of said new ovulation cycle in said female subject based on said analysis.

Further in accordance with a preferred embodiment of the present invention and further including checking if each daily temperature measurement of said oral temperature measurements is within a predetermined temperature range of said previous average temperature and within said running average temperature.

Still further in accordance with a preferred embodiment of the present invention and also including cancelling said daily temperature measurement if said daily oral temperature measurement of said oral temperature measurements exceeds said predetermined said previous average temperature and said running average temperature, alerting said female subject that the current daily temperature exceeds said predetermined temperature range, repeating said daily oral temperature measurement to obtain a new daily oral temperature measurement, calculating a further average current oral temperature, analyzing said further set of current oral temperature measurements with previously stored oral temperature measurements and predicting a commencement of a new ovulation cycle of said female subject based on said analysis.

Typically, the predetermined temperature range is 0.15-0.45° C.

Additionally in accordance with a preferred embodiment of the present invention said oral temperature measurements are measured over a 12-day time interval.

Furthermore in accordance with a preferred embodiment of the present invention includes predicting the sex of a conceived child.

In accordance with a preferred embodiment of the present invention an ovulation sensing and analyzing system for measuring oral temperatures of a female subject comprising an oral temperature sensing system, said oral temperature sensing system including at least one sensing device and a remote computing platform. The at least one sensing device includes an oral temperature measuring sensor to measure said oral temperatures and said at least one sensing device communicates said measured temperatures to said remote computing platform and said remote computing platform analyzes said oral temperature measurements to predict and display on an output display device a date of a next ovulation cycle of said female subject.

In accordance with another preferred embodiment of the present invention includes a method for measuring an oral temperature of a female subject during an ovulation cycle of a female subject and includes providing an oral temperature sensor, measuring said oral temperature with said oral temperature sensor, comparing and analyzing said measured oral temperatures and previously stored oral temperatures of said female subject and predicting a date of a next ovulation cycle of said female subject.

Further in accordance with a preferred embodiment of the present invention the system and method is noninvasive.

Further in accordance with a preferred embodiment of the present invention includes displaying said next ovulation cycle on a display device.

Still further in accordance with a preferred embodiment of the present invention the step of comparing and analyzing includes an iterative method for predicting said commencement of an ovulation cycle in said female subject.

Additionally in accordance with a preferred embodiment of the present invention the step of comparing and analyzing includes a learning method for predicting said commencement of an ovulation cycle in said female subject.

Furthermore in accordance with a preferred embodiment of the present invention the step of comparing and analyzing includes a feedback method for predicting said commencement of an ovulation cycle in said female subject.

Further in accordance with a preferred embodiment of the present invention the at least one sensing device further comprising a control unit, said control unit includes an A/D device for converting measured oral temperatures analog signals to digital signals and a first transceiver for modulation and communicating said digital signals to said remote computing device.

Still further in accordance with a preferred embodiment of the present invention the oral temperature sensor is selected from any one of a group consisting of a thermometer, an oral thermocouple device, a saliva-sensitive temperature measuring device, a digital thermometer, a thermometer sensitive to a predetermined range of temperatures or any combination thereof.

Additionally in accordance with a preferred embodiment of the present invention the computing platform includes a transceiver for demodulating said communicated signals, a processor for processing said demodulated signals and an output device for displaying said processed signals.

Furthermore in accordance with a preferred embodiment of the present invention further includes at least one switch device for activating said at least one sensing device to commence oral temperatures measurements and to communicate said oral temperature measurements to said remote computing platform.

Further in accordance with a preferred embodiment of the present invention the oral temperature sensing system is selected from any one of a group consisting of a smart cellular phone, a laptop, a PDA device or any combination thereof.

Still further in accordance with a preferred embodiment of the present invention the remote computing platform analyzes and displays said analyzed data in graphical form.

Additionally in accordance with a preferred embodiment of the present invention the at least one sensing device includes a USB connection interface for coupling said at least one sensing device to an external storage and analysis device.

Furthermore in accordance with a preferred embodiment of the present invention the oral temperature sensing system detects and measures temperature changes in a range of 0.15-0.45° C.

Further in accordance with a preferred embodiment of the present invention includes an audio sensor for sensing audio data for measuring environmental noise.

Still further in accordance with a preferred embodiment of the present invention includes a virus detector for detecting virus data.

Additionally in accordance with a preferred embodiment of the present invention includes a sensor for detecting hormonal changes in a body.

Furthermore in accordance with a preferred embodiment of the present invention includes a body fat-muscle relation detecting sensor for sensing the body fat-muscle relation data.

Further in accordance with a preferred embodiment of the present invention includes a sensor for sensing and measuring ambient temperature.

Still further in accordance with a preferred embodiment of the present invention includes a wristwatch including a sensor for detecting and storing body temperatures.

Additionally in accordance with a preferred embodiment of the present invention includes a key ring for carrying said temperature sensing device.

Preferably, the at least one sensing device is selected from any one of a group consisting of a temperature sensor, a microbiological sensor, a gas analysis sensor, a pH sensor, an electroconductivity sensor, a physiological sensor, a biometric sensor or any combination thereof.

### BRIEF DESCRIPTIONS OF THE DRAWINGS

Preferred embodiments of the current invention are described hereinbelow with reference to the following drawings:

FIG. 1 shows a general view of a female subject measuring her oral temperature and communicating the measurement to a computing platform, in accordance with a preferred embodiment of the present invention;

FIG. 2 shows the components included in a mobile oral temperature sensing system including a sensing apparatus and the computing platform, in accordance with a preferred embodiment of the present invention;

FIG. 3 shows a method of measuring the oral temperature including the steps of oral temperature measurements, communicating the measurements to the computing platform and displaying the results on an output device, in accordance with a preferred embodiment of the present invention;

FIG. 4 shows a method of recording, storing and analyzing the oral temperature measurements over an ovulation cycle of the female subject in order to predict a next ovulation cycle and the sex of a conceived child, in accordance with a preferred embodiment of the present invention;

FIG. 5 shows a method of obtaining a monthly summary of the measured oral temperatures in order to obtain an infertility report, in accordance with a preferred embodiment of the present invention, and

FIG. 6 shows typical results for an oral temperature measurement for determining the sex of a child, in accordance with a preferred embodiment of the present invention.

### DETAILED DESCRIPTION OF A PREFERRED EMBODIMENT OF THE PRESENT INVENTION

It is herein appreciated that the embodiments of the invention are directed towards exploiting the female menstrual cycle in typical woman. Such a cycle is generally composed of the following phases:
1. An approximately 10-15 day infertile time interval.
2. An approximately 4-6 day preovulation time interval.
3. An approximately 10 day post ovulation time interval.
It is herein acknowledged that the most optimal time intervals for fertility are during the approximately 4-6 day preovulation time interval and the ovulation day itself referred to above. The closer to the day of ovulation, the greater the chance there is of a female child being conceived. The further from the day of ovulation, the greater is the chance of a male child being conceived.
It is herein acknowledged that the most optimal time intervals for intercourse without ensuing conception, in other words, natural contraception, is the approximately 10 day post ovulation time interval.
The above biological phenomena and current knowledge are herein utilized by the present invention embodiments of which are described herein.

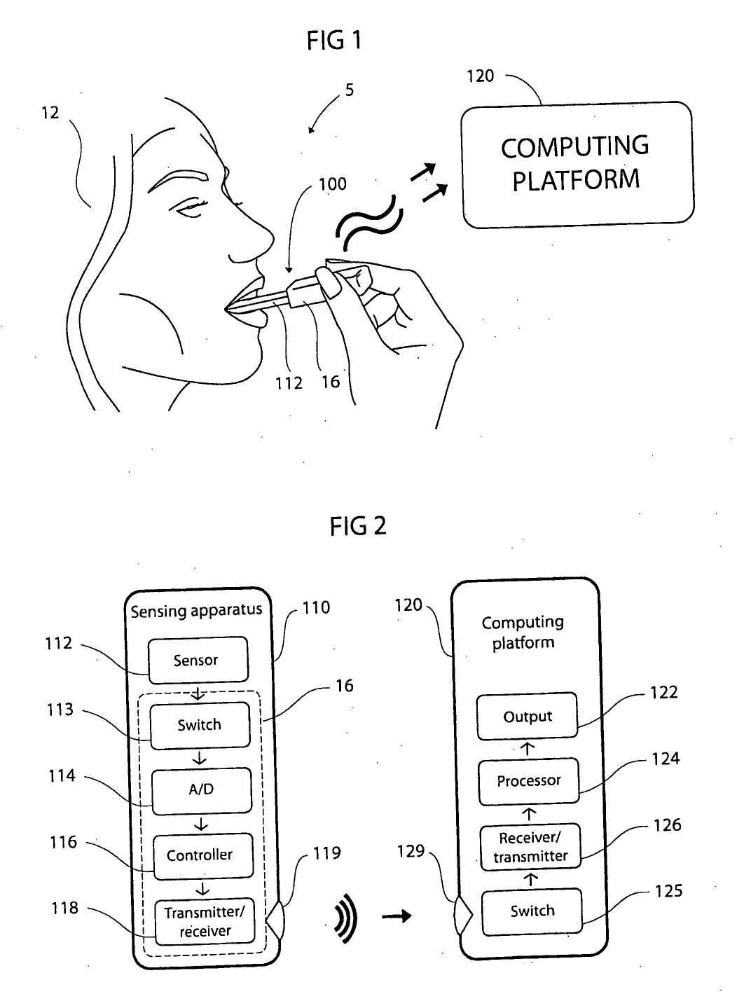

Reference is now made to FIGS. 1 and 2, which show an ovulation sensing and analyzing system 5 including a mobile oral temperature sensing system 100 for sensing, measuring and communicating with a remote computing platform, oral temperature measurements of a female subject 12 during ovulation, in accordance with a preferred embodiment of the present invention. The mobile oral temperature sensing system 100 includes a sensing apparatus 110 and computing platform 120. The sensing apparatus 110 includes at least one oral temperature measuring sensor 112, such as an oral thermometer, which is inserted into the mouth of the female subject 12 and a control unit 16 associated with the oral temperature measuring sensor 112. The sensing apparatus 110 communicates the measured oral temperatures to the computer platform 120.

The control unit 16 includes an A/D device 114 for converting the analog temperature measurements received from the temperature measuring sensor 112 to digital signals, a controller 116 for collating and formatting the digital signals into the required format for communications and a first transceiver 118 for typically modulating and wirelessly communicating the digital signals to a computing platform 120, via an antenna 119.

The control unit 16 typically also includes a first switch device 113 for activating the ovulation sensing and analyzing device 5 on commencement of the temperature measurements and for communicating the temperature measurements in the form of modulated digital signals to the computing platform 120.

Typically, the digital temperature measurements are wirelessly communicated to the computing platform 120 by means of an RF transceiver, Bluetooth, IR transceiver and an audio transceiver. It is appreciated that a wired device, such as a wire connection may also transfer the digital temperature measurements to the computing platform 120.

Additionally or alternatively, the temperature sensing apparatus 110 may include an interface unit, such as a USB connection, for coupling the temperature sensing apparatus 110 to an external non-remote storage and analysis device.

The computing platform 120 includes a second antenna 129 and a second transceiver 126 for receiving the digital signals communicated from the sensing apparatus 110. The second transceiver 126 demodulates the communicated signals and forwards the demodulated signals to a processor 124 for recording, storing and analyzing the measured temperatures. The processor 124 preferably also records the date and time of the temperature measurements as a function of time and date over a predetermined time interval, such as the typical 12-day ovulation time interval of the female subject. In addition, the processor 124 processes and analyzes the signals in order to allow the female subject to observe the measured oral temperature.

Additionally or alternatively, the ovulation sensing and analyzing device 5 is activated by a further switch device 125 located on the computing platform 120 thereby allowing the computer platform 120 to communicate a command signal to the sensing apparatus 110 to commence the temperature measurements.

Additionally or alternatively, the oral temperature sensor 112 includes a saliva-sensitive tip so that measurement of the oral temperature commences on insertion of the thermometer into the mouth.

It is further appreciated that the ovulation sensing and analyzing device 5 is activated by the oral temperature sensor 112 sensing an oral temperature within a predetermined oral temperature range.

In the processor 124, the currently measured oral temperatures are compared with previously measured, recorded and stored oral temperatures, allowing the female subject 10 to observe and note the changes in the oral temperature over a predetermined time.

Additionally or alternatively, the current and previous oral temperature measurements are displayed on an output display unit 122 for immediate observation and study. These measurements may also be forwarded to an output unit, such as a printer for off-line study thereof, for example, in graphical form.

It is appreciated that the analysis and comparison of the oral measured temperature data with previous oral measured temperatures presents statistical information for predetermining and predicting the date of the next ovulation cycle of the female subject 10.

A typical statistical method for predetermining and predicting the next date of ovulation includes a time-dependent regression method, such as Cox's Proportional Hazard Regression or a Time Series Analysis.

It also is appreciated that the oral temperature sensing device 110 includes communication devices such as a smart cellular phone, a laptop and a PDA device.

It also is appreciated that the ovulation sensing and analyzing device 5 is included in a communication device such as a smart cellular phone, a laptop and a PDA device.

It is further appreciated that the temperature measuring sensor 110 may also include a display, such as a color display, for displaying the current temperature measurements.

The temperature sensing apparatus 110 may also include a processor for analyzing the temperature measurements and displaying, for example, the temperature measurements in graphical form.

It is further appreciated that the temperature sensing device 110 device is preferably of sufficient sensitivity so as to measure a change in the oral temperature of approximately 0.15-0.45° C. over the oral temperature.

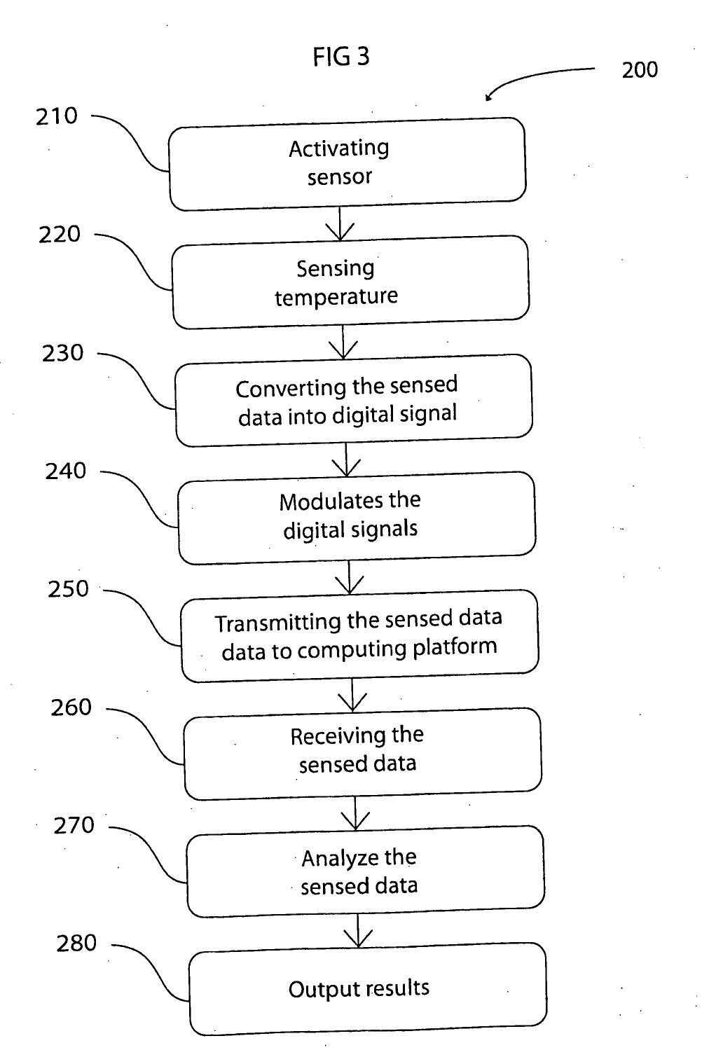

Reference is now made to FIG. 3, which shows a method for measuring the oral temperature and communicating the temperature measurement to the computing platform 120 for storage and analysis, in accordance with the preferred embodiment of the present invention.

In step 210, the temperature sensor 112 is activated by the switch 113 located in the control unit 16 or by the switch 125 located in the computing platform 120. In the latter case, the computing platform 120 communicates a command signal to the sensing apparatus 110 to commence the temperature measurements, via the antenna 129.

In step 220, the oral temperature is sensed and measured.

In step 230, the A/D device 114 converts the measured temperature data into digital signals and in step 240, the digital signals are modulated in preparation for transmission by the transceiver 118 to the computing platform 120.

In step 250, the modulated data is transmitted to the computing platform 120 and in step 260, the data is received at the computing platform 120.

In steps 270 and 280, the data is analyzed and displayed, respectively, as discussed hereinbelow, in accordance with the requirements of the female subject 10.

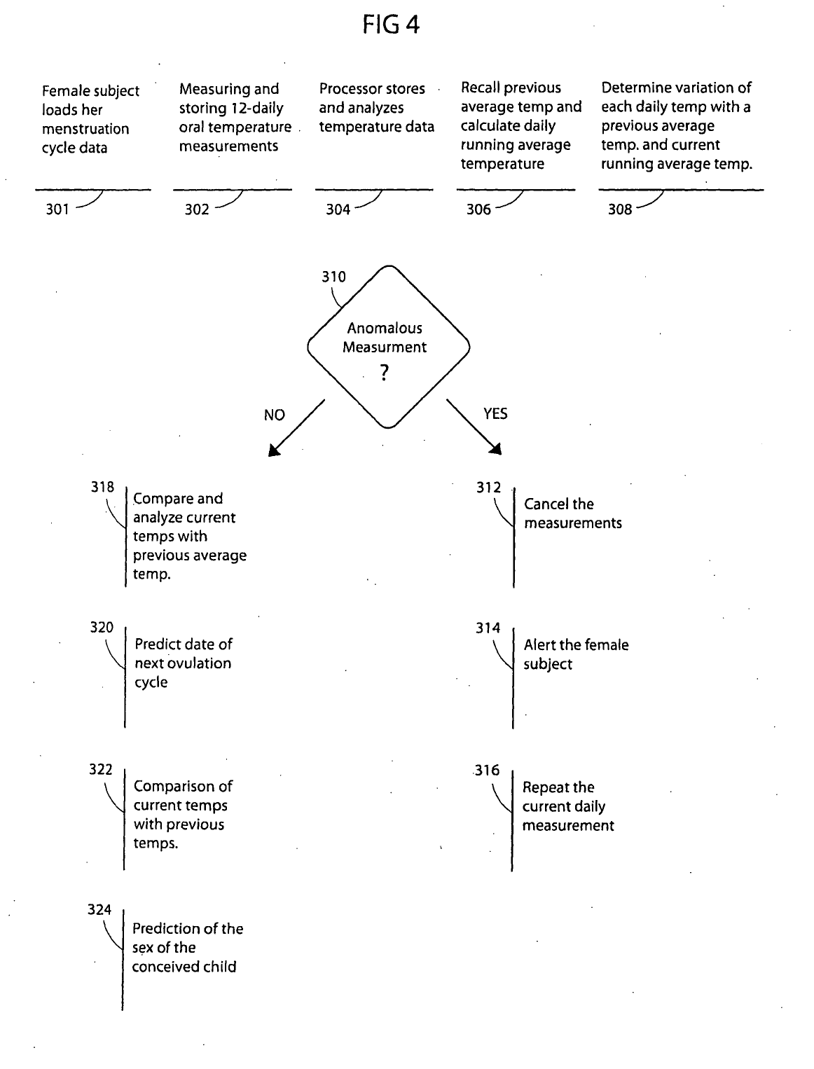

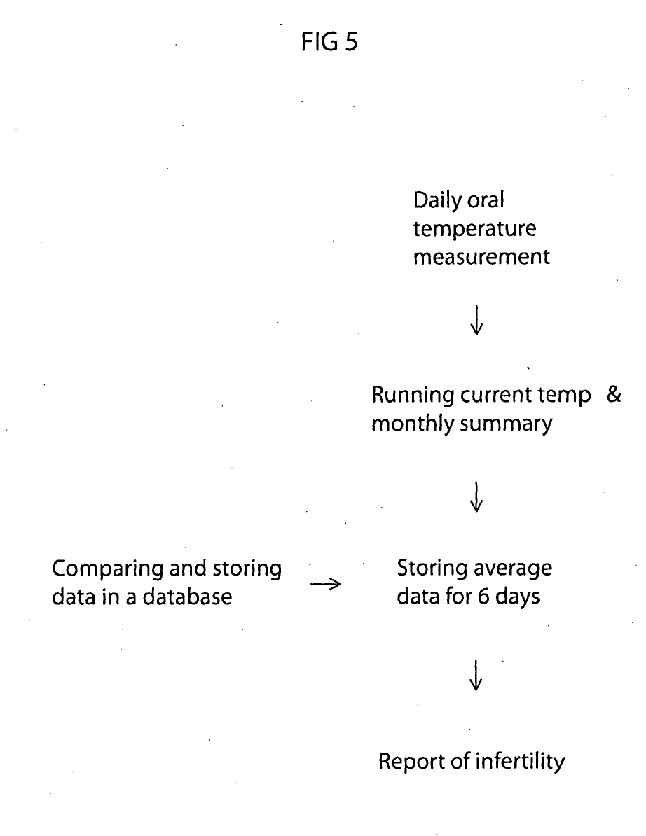

Reference is now made to FIG. 4, which shows a flow chart 300 of a typical iterative and learning procedure carried by the processor 124 for providing the female subject with an analysis of the oral temperature measurements. The measurements are performed over a predetermined time interval and typically, for example, a 12-day time interval, during the female subject's ovulation cycle. On comparison of the current temperature measurements with previously stored oral temperature measurements, the female subject is able to predict the date of commencement of the next ovulation period as well as predicting the sex of a conceived child.

As described hereinabove, the processor 124 receives from the temperature sensing apparatus 110, the oral temperature measurements typically taken over a 12-day time interval by the mobile oral temperature sensing system 100. The processor 124 records, stores and analyzes the 12-daily oral temperature measurements of the female subject. Preferably, the oral measurements are conducted in the morning shortly after the female subject has awoken from her nightly sleep.

In step 301, the female subject loads her menstruation cycle data, including the date and length of her previous menstruation cycles into the processor 124. In step 302, following the menstruation period and during the ovulation period, the female subject performs typically, for example, 12-daily oral temperature measurements. After each daily measurement, the measurement is communicated to the computing platform 120 where the processor 124 records, stores and analyzes the temperature data (step 304).

In step 306, the processor 124, based on the received current daily oral temperature measurements, compares each current daily oral temperature measurement with at least one average oral temperature which has been previously calculated from a previous set of oral temperature measurements measured during a previous complete ovulation cycle and calculates a first daily variation. In addition, the processor 124 compares the daily temperature with a running average of the current daily temperature measurements and calculates a second daily variation.

It is appreciated that the daily oral temperature measurements and/or average oral temperatures are also available on external databases and may be used for comparing and checking the current temperature measurements.

In step 308, during the 12-day ovulation period, the processor 124 checks if either or both the first and second daily variations of the current daily measurement is within a temperature range of between approximately 0.15-0.45° C. of the average temperature and the average running temperature, respectively. If on a particular day, the processor 124 determines that either the first variation and/or the second variation exceeds the temperature range of 0.15-0.45° C. of the previous average and/or the running average temperature, respectively, the processor 124 decides that this daily measurement is anomalous (step 310) and the current daily measurement is cancelled (step 312). The female subject is alerted to the anomalous measurement by receiving a relevant warning message which is displayed on the output display unit 122 (step 314) and she is requested to repeat this daily oral temperature measurement (step 316).

If the measurements are not anomalous (step 310), the processor 124 compares the current oral temperature measurements against previously stored oral temperatures and basal body temperatures (step 318) and based on a statistical analysis, provides the female subject with a prediction of the date of commencement of the next ovulation cycle (step 320).

It is appreciated that if the new daily measurement also exceeds the temperature range of 0.15-0.45° C., the oral temperature measurement is repeated.

A typical statistical analysis for predetermining and predicting the next date of ovulation includes a time-dependent regression method, such as Cox's Proportional Hazard Regression or a Time Series Analysis.

In addition, based on previously stored data of the day of conception as well as the oral temperature measurements and prestored temperature measurements, the processor provides the female subject with prediction as to the sex of the conceived child (steps 322 and 324).

Reference is now made to FIG. 5, which shows that the processor 124 also stores the average daily temperature and provides a monthly oral temperature report. Furthermore, based on a six-day average oral temperature, the processor 124 provides an infertility report to assist the female subject in birth-control.

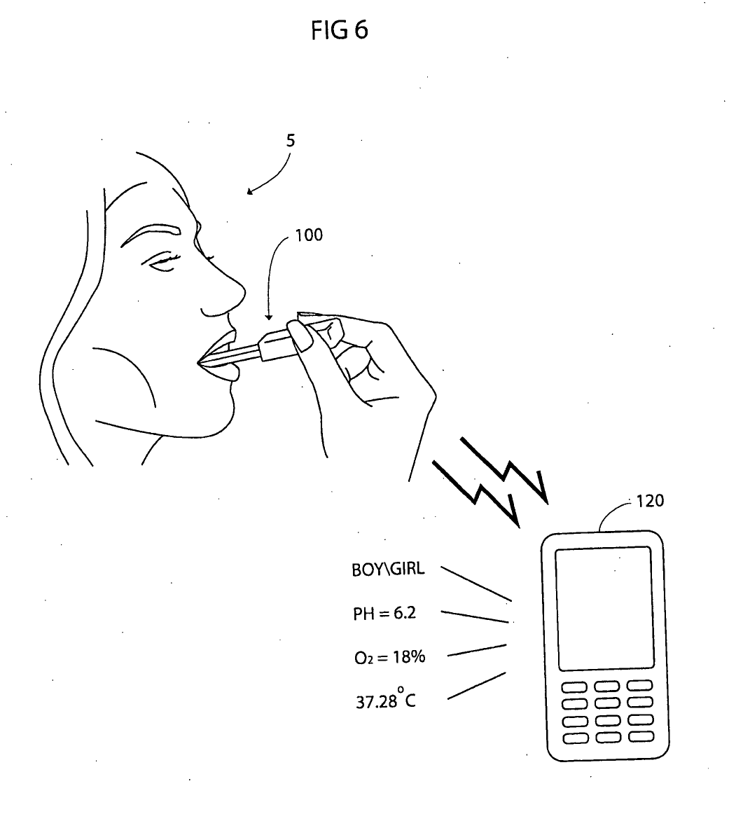

Reference is now made to FIG. 6 which shows typical results for an oral temperature measurement for determining the sex of a child, in accordance with a preferred embodiment of the present invention. During the measure of the oral temperature, the female subject also obtains pH and O₂ measurements and deduces the sex of a conceived child.

In the foregoing description, embodiments of the invention, including preferred embodiments, have been presented for the purpose of illustration and description. They are not intended to be exhaustive or to limit the invention to the precise form disclosed. Obvious modifications or variations are possible in light of the above teachings. The embodiments were chosen and described to provide the best illustration of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A method for predicting a commencement of a new ovulation cycle in a female subject comprising:
recording the commencement and termination of a menstruation cycle;
measuring on a daily basis, an oral temperature of said female subject during a current ovulation cycle following said termination of said menstruation cycle;
calculating a current running average of the daily temperature measurements;
comparing each daily measurement with at least one previous average oral temperature calculated from at least one previous ovulation cycle;
storing said current daily measured oral temperatures;
storing said current daily running average temperatures of current measured oral temperatures;
calculating a current average oral temperature from said current daily oral temperature measurements;
storing said current average oral temperature;
comparing and analyzing said current daily temperature with said at least one previous average temperature;
predicting said commencement of said new ovulation cycle in said female subject based on said analysis.

2. The method for predicting a commencement of a new ovulation cycle in a female subject in accordance with claim 1 and further comprising checking if each daily temperature measurement of said oral temperature measurements is within a predetermined temperature range of said previous average temperature and within said running average temperature.

3. The method for predicting the commencement of a new ovulation cycle according to claim 2 and further comprising:
cancelling said daily temperature measurement if said daily oral temperature measurement of said oral temperature measurements exceeds said predetermined said previous average temperature and said running average temperature alerting said female subject that the current daily temperature exceeds said predetermined temperature range;
repeating said daily oral temperature measurement to obtain a new daily oral temperature measurement;
calculating a further average current oral temperature;
analyzing said further set of current oral temperature measurements with previously stored oral temperature measurements, and
predicting a commencement of a new ovulation cycle of said female subject based on said analysis.

4. The method for predicting a commencement of a new ovulation cycle in a female subject in accordance with any one of claims 1-3 said oral temperature measurements are measured over a 12-day time interval.

5. The method for predicting the commencement of an ovulation cycle according to any one of claims 1-4 and further comprising predicting the sex of a conceived child.

6. An ovulation sensing and analyzing system for measuring oral temperatures of a female subject comprising an oral temperature sensing system, said oral temperature sensing system comprising:
at least one sensing device, and
a remote computing platform,
wherein said at least one sensing device includes an oral temperature measuring sensor to measure said oral temperatures and said at least one sensing device communicates said measured temperatures to said remote computing platform and said remote computing platform analyzes said oral temperature measurements to predict and display on an output display device a date of a next ovulation cycle of said female subject.

7. The ovulation sensing and analyzing system according to claim 6 and said at least one sensing device further comprising a control unit, said control unit comprising:
an A/D device for converting measured oral temperatures analog signals to digital signals, and
a first transceiver for modulation and communicating said digital signals to said remote computing device.

8. The ovulation sensing and analyzing system according to any one of claims 6 or 7 said oral temperature sensor is selected from any one of a group consisting of a thermometer, an oral thermocouple device, a saliva-sensitive temperature measuring device, a digital thermometer, a thermometer sensitive to a predetermined range of temperatures or any combination thereof.

9. The ovulation sensing and analyzing system according to claim 6 said computing platform comprising:
a transceiver for demodulating said communicated signals;
a processor for processing said demodulated signals, and
an output device for displaying said processed signals.

10. The ovulation sensing and analyzing system according to any one of claims 6-9 and further comprising at least one switch device for activating said at least one sensing device to commence oral temperatures measurements and to communicate said oral temperature measurements to said remote computing platform.

11. The ovulation sensing and analyzing system according to any one of claims 6-10 said oral temperature sensing system is selected from any one of a group consisting of a smart cellular phone, a laptop, a PDA device or any combination thereof.

12. The ovulation system according to any one of claims 6-11 said remote computing platform analyzes and displays said analyzed data in graphical form.

13. The ovulation sensing and analyzing system according to any one of claims 6-12 said at least one sensing device includes a USB connection interface for coupling said at least one sensing device to an external storage and analysis device.

14. The ovulation sensing and analyzing system according to any one of claims 6-13 said oral temperature sensing system detects and measures temperature changes in a range of 0.15-0.45° C.

15. The ovulation sensing and analyzing system according to any one of claims 6-14 and further comprising an audio sensor for sensing audio data for measuring environmental noise.

16. The ovulation sensing and analyzing system according to any one of claims 6-14 and further comprising a virus detector for detecting virus data.

17. The ovulation sensing and analyzing system according to any one of claims 6-14 and further comprising a sensor for detecting hormonal changes in a body.

18. The ovulation sensing and analyzing system according to any one of claims 6-14 and further comprising a body fat-muscle relation detecting sensor for sensing the body fat-muscle relation data.

19. The ovulation sensing and analyzing system according to any one of claims 6-14 and further comprising a sensor for sensing and measuring ambient temperature.

20. The ovulation sensing and analyzing system according to any one of claims 6-14 and further comprising a wristwatch including a sensor for detecting and storing body temperatures.

21. The ovulation sensing and analyzing system according to any one of claims 6-14 and further comprising a key ring for carrying said temperature sensing device.

22. A method for measuring an oral temperature of a female subject during an ovulation cycle of a female subject, comprising:
providing an oral temperature sensor;
measuring said oral temperature with said oral temperature sensor;
comparing and analyzing said measured oral temperatures and previously stored oral temperatures of said female subject, and
predicting a date of a next ovulation cycle of said female subject based on said analysis.

23. The method for measuring an oral temperature of a female subject during an ovulation cycle of a female subject according to claim 22 and comprising displaying said next ovulation cycle on a display device.

24. The method for measuring an oral temperature of a female subject during an ovulation cycle of a female subject according to any one of claims 22 and 23 and comprising providing a control unit, said control unit comprising:
an A/D device for converting measured oral temperatures to digital signals, and
a first transceiver for modulation and communicating said digital signals to said a computing device.

25. The method for measuring an oral temperature of a female subject during an ovulation cycle of a female subject according to any one of claims 22-24 and comprising providing an oral temperature sensor selected from any one of a group consisting of a thermometer, an oral thermocouple device, a saliva-sensitive temperature measuring device, a digital thermometer, a thermometer sensitive to a predetermined range of temperatures or any combination thereof.

26. The method for measuring an oral temperature of a female subject during an ovulation cycle of a female subject according to any one of claims 22-25 and further comprising said remote computing platform receiving and demodulating said communicated signals, processing said demodulated signals, and displaying said processed signals on said display device.

27. The method for measuring an oral temperature of a female subject during an ovulation cycle of a female subject according to any one of claims 22-26 and comprising providing at least one switch device for activating said oral temperature sensing device to commence oral temperatures and to communicate said temperature measurements to said remote computing platform.

28. The method for measuring an oral temperature of a female subject during an ovulation cycle of a female subject according to any one of claims 22-27 and further comprising selecting said oral temperature sensing system from any one of a group consisting of a smart cellular phone, a laptop, a PDA device or any combination thereof

29. The method for measuring an oral temperature of a female subject during an ovulation cycle of a female subject according to any one of claims 22-28 and further comprising providing said remote computing platform for analyzing and displaying said analyzed data in graphical form.

30. The method for measuring an oral temperature of a female subject during an ovulation cycle of a female subject according to any one of claims 22-29 and further comprising a USB connection interface located on said sensing device for coupling said sensing device to an external storage and analysis device.

31. The method for measuring an oral temperature of a female subject during an ovulation cycle of a female subject according to any one of claims 22-30 and further comprising providing said oral temperature sensing device for detecting and measuring temperature changes in a range of 0.15-0.45° C.

32. The method for measuring an oral temperature of a female subject during an ovulation cycle of a female subject according to claim any one of claims 22-31 and further comprising providing an audio sensor for sensing audio data for measuring environmental noise.

33. The method for measuring an oral temperature of a female subject during an ovulation cycle of a female subject according to claim any one of claims 22-31 and further comprising providing a virus detector for detecting virus data.

34. The method for measuring an oral temperature of a female subject during an ovulation cycle of a female subject according to claim any one of claims 22-31 and further comprising providing a sensor for detecting hormonal changes in a body.

35. The method for measuring an oral temperature of a female subject during an ovulation cycle of a female subject according to claim any one of claims 22-31 and further comprising providing a sensor for sensing body fat-muscle relation sensor and the sensed data is the body fat-muscle relation.

36. The method for measuring an oral temperature of a female subject during an ovulation cycle of a female subject according to claim any one of claims 22-31 and further comprising providing a body fat-muscle relation detecting sensor for sensing the body fat-muscle relation data.

37. The method for measuring an oral temperature of a female subject during an ovulation cycle of a female subject according any one of claims 22-31 and further comprising providing a wristwatch including a sensor for detecting and storing body temperature.

38. The method for measuring an oral temperature of a female subject during an ovulation cycle of a female subject according to any one of claims 22-31 and further comprising providing a key ring for carrying said temperature sensing device.

39. A method for predicting a commencement of an ovulation cycle in a female subject in accordance with any one of claims 1-5 said comparing and analyzing includes at least one iterative step for predicting said commencement of said new ovulation cycle in said female subject.

40. A method for predicting a commencement of an ovulation cycle in a female subject in accordance with any one of claims 1-5 said comparing and analyzing includes at least one learning step for predicting said commencement of said new ovulation cycle in said female subject.

41. A method for predicting a commencement of an ovulation cycle in a female subject in accordance with any one of claims 1-5 said comparing and analyzing includes at least one feedback step for predicting said commencement of said new ovulation cycle in said female subject.

42. The ovulation sensing and analyzing system according to any one of claims 6-21 said at least one sensing device is selected from any one of a group consisting of a temperature sensor, a microbiological sensor, a gas analysis sensor, a pH sensor, an electroconductivity sensor, a physiological sensor, a biometric sensor, a miniature stills camera or video imaging device with or without illumination means such as but not limited to an LED, a fiber optic device, or any combination thereof.

43. The ovulation sensing and analyzing system according to any one of claims 6-21 wherein at least one of said sensors is noninvasive.

44. The method for predicting a commencement of a new ovulation cycle in a female subject in accordance with any one of claims 1-5 is noninvasive.

45. The method for measuring an oral temperature of a female subject during an ovulation cycle of a female subject in accordance with any one of claims 17-31 is noninvasive.

46. The method for predicting a commencement of a new ovulation cycle in a female subject in accordance with any one of claims 1-5 and said predetermined temperature range is 0.15-0.45° C.
